(19) United States
(12) Patent Application Publication
(10) Pub. No.: US 2026/0098243 A1
(43) Pub. Date: Apr. 9, 2026

(54) DURA MATER-DERIVED STEM CELLS AND METHOD FOR PRODUCING THE SAME

(71) Applicant: THE CATHOLIC UNIVERSITY OF KOREA INDUSTRY-ACADEMIC COOPERATION FOUNDATION, Seoul (KR)

(72) Inventors: Sun Hwa PARK, Seoul (KR); Sung Won KIM, Seoul (KR); Seung Ho YANG, Seoul (KR); Jung Eun LEE, Gyeonggi-do (KR); Hyun Ji LEE, Gyeonggi-do (KR)

(21) Appl. No.: 19/350,367

(22) Filed: Oct. 6, 2025

(30) Foreign Application Priority Data
Oct. 7, 2024 (KR) 10-2024-0135690
Sep. 29, 2025 (KR) 10-2025-0140717

(51) Int. Cl. C12N 5/0797

(52) U.S. Cl. CPC C12N 5/0623; C12N 2500/14; C12N 2500/16; C12N 2500/32; C12N 2500/34; C12N 2500/38; C12N 2500/40; C12N 2500/84

(57) ABSTRACT

A dura mater-derived stem cell and a method of producing the same according to the present invention establish a method of isolating and producing stem cells from the dura mater by adding serum or a serum substitute, which is a culture supplement, to a conventionally used medium. The dura mater-derived stem cells produced by the production method of the present invention were confirmed to have excellent differentiation potency into mesenchymal and neurons and exhibit physical characteristics that enable them to maintain high cell viability when transplanted into cell therapeutic agents. Furthermore, they have been confirmed to possess inherent expression marker characteristics so that they can be effectively used in various applications including cell therapeutic agents.

### CROSS-REFERENCE TO RELATED APPLICATION

This application claims priority to and the benefit of Korean Patent Application No. 10-2025-0140717, filed on Sep. 29, 2025, the disclosure of which is incorporated herein by reference in its entirety.

### BACKGROUND

### Field of the Invention

The present invention relates to dura mater-derived stem cells and a method for producing the same.

### Discussion of Related Art

Stem cells, possessing self-renewal capability and multipotency, have been an important research target in regenerative medicine and cell therapy. In particular, technologies for inducing stem cells from somatic cells have drawn attention as a means to overcome the limitations of existing adult stem cells by enabling differentiation into various tissues. However, existing stem cell induction technologies are limited by complex manipulation procedures, low induction efficiency, and ethical concerns.

Accordingly, research is actively underway on methods for obtaining or deriving stem cells from new tissues or regions. In particular, attempts to isolate and derive stem cells from tissues that are relatively easily obtained through injury or surgical procedures have been reported. These technological approaches could contribute to securing a stable source of stem cells and expanding their clinical applicability.

Meanwhile, the dura mater is a dense fibrous membrane located at the outermost layer of the three meninges surrounding the central nervous system. The dura mater is composed primarily of collagen fibers, and thus it possesses high mechanical strength and serves to protect the brain and spinal cord from external impacts. However, its practical utility is limited because it is often discarded after surgical removal.

Despite this technological background, few cases have been reported on the production of stem cells from the dura mater and the characterization of their characteristics.

### SUMMARY OF THE INVENTION

### Prior Art Documents

### Patent Documents

(Patent Document 1) Korean Patent Application Publication No. 10-2022-0033444

### DISCLOSURE

### Technical Problem

An object of the present invention is to provide a dura mater-derived stem cell negatively expressing one or more markers selected from the group consisting of CD14, CD19, CD34, and HLA-DR and positively expressing one or more markers selected from the group consisting of CD44, CD73, CD90, CD105, and CD166.

Another object of the present invention is to provide a medium composition for producing dura mater-derived stem cells, comprising as active ingredients:
a basal medium composition including one or more active ingredients selected from the group consisting of amino acids, vitamins, minerals, glucose, and cell culture supplements; and
serum or a serum substitute.

Still another object of the present invention is to provide a method of producing dura mater-derived stem cells, comprising the step of producing dura mater-derived stem cells by chopping isolated dura mater tissue and culturing the chopped isolated dura mater tissue in the medium composition.

A further object of the present invention is to provide a kit for producing dura mater-derived stem cells, comprising the medium composition for producing dura mater-derived stem cells and an instruction manual.

However, technical problems to be solved in the present invention are not limited to the above-described problems, and other problems which are not described herein will be fully understood by those of ordinary skill in the art from the following descriptions.

### Technical Solution

The present invention provides a dura mater-derived stem cell negatively expressing one or more markers selected from the group consisting of CD14, CD19, CD34, and HLA-DR and positively expressing one or more markers selected from the group consisting of CD44, CD73, CD90, CD105, and CD166.

In one embodiment of the present invention, the dura mater-derived stem cell may further positively express one or more selected from the group consisting of CD106 and CD9, but is not limited thereto.

In one embodiment of the present invention, the dura mater-derived stem cell may have an average diameter of 14 μm to 18 μm, but is not limited thereto.

In one embodiment of the present invention, the dura mater-derived stem cell may differentiate into one or more mesodermal lineages selected from the group consisting of adipocytes, chondrocytes, and osteocytes, or may differentiate into a neural lineage, but is not limited thereto.

In one embodiment of the present invention, a cell of the neural lineage may positively express one or more selected from the group consisting of neurofilament (NF), neuron-specific class III β-tubulin (Tuj1), glial fibrillary acidic protein (GFAP), microtubule-associated proteins (MAP2), and neuronal nuclei (NeuN), but is not limited thereto.

In one embodiment of the present invention, the dura mater-derived stem cell may be passaged 1 to 10 times, but is not limited thereto.

In one embodiment of the present invention, a cell of the neural lineage may be characterized by one or more selected from the following, but is not limited thereto:
increased axon and dendrite formation in neurons; and
increased protrusion density and formation in neurons.

The present invention provides a medium composition for producing dura mater-derived stem cells, comprising as active ingredients:
a basal medium composition including one or more active ingredients selected from the group consisting of amino acids, vitamins, minerals, glucose, and cell culture supplements; and
serum or a serum substitute.

In one embodiment of the present invention, the amino acid may be one or more selected from the group consisting of L-alanine, L-arginine, L-asparagine, L-aspartic acid, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine:
the vitamin may be one or more selected from the group consisting of ascorbic acid, biotin, choline chloride, folic acid, inositol, nicotinamide, calcium pantothenate, pyridoxine-HCl, riboflavin, thiamine-HCl, and vitamin B12;
the inorganic salt may be one or more selected from the group consisting of sodium chloride, potassium chloride, calcium chloride, magnesium sulfate, sodium dihydrogen phosphate, and sodium bicarbonate;
the glucose may be D-glucose; and
the cell culture supplement may be adenine sulfate, but is not limited thereto.

In one embodiment of the present invention, the serum may be one or more selected from the group consisting of fetal bovine serum (FBS), newborn calf serum (NCS), calf serum (CS), horse serum, goat serum, donor bovine serum (DBS), and human serum; and
the serum substitute may be one or more selected from the group consisting of a growth factor reduced supplement, a chemically defined lipid concentrate, an insulin-transferrin-selenium supplement, a B27 supplement, an N2 supplement, a serum substitute supplement, and a xeno-free serum replacement, but is not limited thereto.

The present invention provides a method of producing dura mater-derived stem cells, comprising the step of producing dura mater-derived stem cells by chopping isolated dura mater tissue and culturing the chopped dura mater tissue in the medium composition for producing dura mater-derived stem cells.

The present invention provides a kit for producing dura mater-derived stem cells, comprising the medium composition for producing dura mater-derived stem cells and an instruction manual.

In one embodiment of the present invention, the instruction manual may teach the production method, but is not limited thereto.

Additionally, the present invention provides a use for producing dura mater-derived stem cells of a composition comprising:
a basal medium composition including one or more active ingredients selected from the group consisting of amino acids, vitamins, minerals, glucose, and cell culture supplements; and
serum or a serum substitute.

### Advantageous Effects

According to the dura mater-derived stem cells and the method for producing the same, a method of isolating and producing stem cells from the dura mater was established by adding serum or a serum substitute, which is a culture supplement, to a conventionally used medium. The dura mater-derived stem cells produced by the production method of the present invention were confirmed to have excellent differentiation potency into mesenchymal cells and neurons and to exhibit physical characteristics that may maintain high cell viability when transplanted as a cell therapeutic agent. Additionally, the cells were confirmed to possess inherent expression marker characteristics, and thus may be effectively utilized in various applications including cell therapeutic agents.

### BRIEF DESCRIPTION OF THE DRAWINGS

FIG. 1A illustrates a method of isolating and producing stem cells from human dura mater.

FIGS. 1B and 1C illustrate the morphology over culture days of stem cells isolated and produced from human dura mater using α-MEM+FBS and IMDM+FBS, respectively, as culture media.

FIG. 2A shows the results of analyzing the proliferative capacity of human dura mater-derived stem cells when cultured from passage 1 to passage 6 in α-MEM or IMDM media treated with FBS or GRO. FIG. 2B shows the results of analyzing the average size of the human dura mater-derived stem cells.

FIG. 3A shows the HLA-DR marker expression of adult stem cells isolated and produced from human dura mater using α-MEM+FBS and IMDM+FBS, respectively, as culture media.

FIGS. 3B and 3C show the results of karyotype analysis and genetic stability analysis performed on the human dura mater-derived stem cells of the present invention.

FIGS. 4A to 4D show the results of marker expression analyses performed during the establishment of medium conditions for producing the human dura mater-derived stem cells of the present invention. FIGS. 4A and 4B show the expression of the MSC-specific markers CD44 and CD166 in adult stem cells isolated and produced from human dura mater using α-MEM+FBS and IMDM+FBS, respectively, as culture media. FIGS. 4C and 4D show the expression of the endothelial-specific marker CD106 and the embryonic stem cell-specific marker CD9.

FIGS. 5A and 5B are in-depth analyses of MSC expression markers of the human dura mater-derived stem cells of the present invention, showing MSC markers confirmed to be negative and MSC markers confirmed to be positive at P8, respectively.

FIGS. 5C to 5E show the results of analyzing expression markers of stem cells isolated and produced from the dura mater of another donor. Each figure shows the results of analyzing MSC markers exhibiting negative or positive expression and the expression of endothelial cell and embryonic stem cell (or induced pluripotent stem cell) markers, as in FIGS. 5A and 5B.

FIG. 6A shows the results of analyzing the proliferative capacity of the dura mater-derived stem cells of the present invention.

FIGS. 6B and 6C show the results of analyzing the post-thaw cell viability of the dura mater-derived stem cells of the present invention.

FIG. 7A shows the results of analyzing the differentiation potency of the dura mater-derived stem cells of the present invention into mesodermal lineages.

FIG. 7B shows the expression levels of NF, Tuj1, GFAP, MAP2, and NeuN before differentiation of the dura mater-derived stem cells of the present invention.

FIG. 7C shows the results obtained by inducing differentiation of the dura mater-derived stem cells of the present invention into a neural lineage for three weeks.

FIG. 7D shows the results of staining and observing NF, Tuj1, GFAP, MAP2, and NeuN in dura mater-derived stem cells, inferior turbinate-derived stem cells, and bone marrow-derived stem cells after inducing their differentiation into neural lineage cells.

### DETAILED DESCRIPTION OF EXEMPLARY EMBODIMENTS

In the present invention, the “dura mater” is a dense fibrous connective tissue located at the outermost layer of the three meninges surrounding the central nervous system, and is composed primarily of cross-arranged collagen fibers and fibroblasts, thereby providing mechanical strength. The dura mater not only protects the brain and spinal cord from external physical impacts, but also possesses functional and structural characteristics that form pathways for cerebrospinal fluid circulation and blood drainage, including the dural venous sinuses.

In the present invention, a “stem cell” refers to a cell population that may proliferate for an extended period through self-renewal and simultaneously possesses differentiation potency (multipotency or pluripotency) toward various cell lineages. Such stem cells are classified according to their developmental origin and differentiation potential into embryonic stem cells, adult stem cells, and induced pluripotent stem cells, and they possess distinct physiological characteristics and application potentials. In particular, mesenchymal stem cells, which are derived from the mesodermal lineage, exhibit differentiation potency into osteocytes, chondrocytes, and adipocytes and perform immunomodulatory functions, and thus are widely utilized in regenerative medicine and the development of cell therapeutic agents.

Sources of stem cells may be isolated from various tissues in the body, including cord blood, bone marrow, adipose tissue, dental pulp, and the dura mater. Depending on the source, the proliferative capacity, differentiation potential, and immunological characteristics of stem cells may differ, and an appropriate tissue may be selected and utilized according to the intended therapeutic application.

Although stem cells may be isolated from various tissues in the body, not all tissues can function as a source of stem cells. This is because the feasibility of maintaining and isolating stem cells depends on the developmental characteristics of the tissue, the cellular microenvironment, and the presence of a stem cell niche. Therefore, in certain tissues, the proliferative capacity or differentiation potency of stem cells may not be secured, and such tissues may not serve as practical sources of stem cells.

Additionally, even stem cells derived from the same tissue may exhibit significantly different proliferation rates, differentiation potency, and surface marker expression patterns depending on the culture conditions. Factors such as the composition of the culture medium, the type and concentration of growth factors, and oxygen tension directly affect the phenotype and functional characteristics of stem cells. Therefore, to secure the stable characteristics of stem cells, it is essential to strictly control not only the tissue origin but also the culture environment.

The inventors of the present invention completed the present invention by obtaining stem cells that, when isolated from dura mater discarded during surgery, exhibit a size suitable for cell transplantation and simultaneously possess differentiation potency while exhibiting unique expression marker characteristics.

Accordingly, the present invention provides a dura mater-derived stem cell, wherein the stem cell negatively expresses one or more markers selected from the group consisting of CD14, CD19, CD34, and HLA-DR, and positively expresses one or more markers selected from the group consisting of CD44, CD73, CD90, CD105, and CD166.

The dura mater-derived stem cells of the present invention may be characterized by negatively or positively expressing specific MSC-specific markers. In this context, the specific MSC-specific markers may exemplarily include CD14 (Cluster of Differentiation 14), CD14 (Cluster of Differentiation 14), CD34 (Cluster of Differentiation 34), HLA-DR (Human Leukocyte Antigen-DR isotype), CD44 (Cluster of Differentiation 44), CD73 (Cluster of Differentiation 73, ecto-5′-nucleotidase), CD90 (Cluster of Differentiation 90, Thy-1), CD105 (Cluster of Differentiation 105, Endoglin), or CD166 (Cluster of Differentiation 166, Activated Leukocyte Cell Adhesion Molecule, ALCAM). The dura mater-derived stem cells of the present invention may negatively express CD14, CD19, CD34, and HLA-DR, and may positively express CD44, CD73, CD90, CD105, and CD166.

In addition, the dura mater-derived stem cells of the present invention were confirmed to additionally express an endothelial cell-specific marker and an embryonic stem cell (or induced pluripotent stem cell)-specific marker. For example, they may express CD106 (Cluster of Differentiation 106, Vascular Cell Adhesion Molecule 1, VCAM-1), an endothelial cell-specific marker, and CD9 (Cluster of Differentiation 9, Tetraspanin-29/Motility-related protein 1), an embryonic stem cell (or induced pluripotent stem cell)-specific marker. Accordingly, in one embodiment of the present invention, the dura mater-derived stem cell may further positively express one or more selected from the group consisting of CD106 and CD9, but is not limited thereto. Taken together, the dura mater-derived stem cells of the present invention may possess unique characteristics by negatively or positively expressing specific MSC-specific markers and additionally expressing endothelial cell- and embryonic stem cell (or induced pluripotent stem cell)-specific markers.

In one embodiment of the present invention, the dura mater-derived stem cell may have an average diameter of 14 μm to 18 μm, but is not limited thereto. For example, it may have an average diameter of 14 μm to 18 μm, 14 μm to 17.6 μm, 14 μm to 17.2 μm, 14 μm to 16.8 μm, 14 μm to 16.4 μm, 14 μm to 16 μm, 14.4 μm to 18 μm, 14.4 μm to 17.6 μm, 14.4μ m to 17.2 μm, 14.4 μm to 16.8 μm, 14.4 μm to 16.4 μm, or 14.4 μm to 16 μm, but is not limited thereto. In particular, in one embodiment of the present invention, the dura mater-derived stem cells of the present invention were confirmed to have an average diameter of 16 μm. Given that a ±10% range of the average value may be considered an acceptable tolerance range for cells, the dura mater-derived stem cells of the present invention may have an average diameter of 14.4 μm to 17.6 μm.

In one embodiment of the present invention, the dura mater-derived stem cell may differentiate into one or more mesodermal lineages selected from the group consisting of adipocytes, chondrocytes, and osteocytes, or may differentiate into a neural lineage, but is not limited thereto.

In one embodiment of the present invention, a cell of the neural lineage may positively express one or more selected from the group consisting of neurofilament (NF), neuron-specific class III β-tubulin (Tuj1), glial fibrillary acidic protein (GFAP), microtubule-associated proteins (MAP2), and neuronal nuclei (NeuN), but is not limited thereto.

In the present invention, “neurofilament (NF)” refers to a class of intermediate filament proteins expressed within the axons of mature neurons. That is, NF is known as a cytoskeletal protein of neurons that primarily performs important functions in the formation and stabilization of axons. The expression of NF reflects the structural stability of neurons and the development of neuronal processes. Accordingly, NF functions as a major marker indicating that neuronal differentiation has progressed and that the neuron has reached a structurally mature stage.

In the present invention, “Tuj1 (neuron-specific class III β-tubulin)” refers to a neuron-specific β-tubulin protein that is expressed during the early stages of neuronal differentiation. It is particularly associated with the formation of neuron-specific structures such as axons and dendrites, and the expression of Tuj1 may function as an early neural differentiation marker indicating that stem cells have begun differentiation into neurons.

In the present invention, “GFAP (glial fibrillary acidic protein)” is an intermediate filament protein that is specifically expressed in astrocytes. During the process in which stem cells differentiate into the nervous system, this marker serves as an indicator of differentiation into glial lineages-particularly glial cells-so it enables identification not only of neuronal differentiation but also of lineage diversification into glial subtypes such as astrocytes, oligodendrocytes, and microglia. Accordingly, GFAP expression is known as an important marker for determining astrocyte differentiation within a cell population.

In the present invention, “MAP2 (microtubule-associated proteins 2)” refers to a protein expressed in the nucleus of mature neurons and is a protein specifically present in dendrites. MAP2 is known as an indicator for determining whether a neuron has progressed beyond a simple progenitor cell stage and has become structurally and functionally mature through the formation of dendrites.

In the present invention, “NeuN (neuronal nuclei)” refers to a protein expressed in the nucleus and cytoplasm of mature neurons and is used to confirm neuronal maturation. When NeuN expression is observed in stem cell-derived neurons, it indicates that the cells have ultimately matured into neurons. Accordingly, NeuN functions as a key marker establishing the identity of differentiated neurons.

In the present invention, “subculture” refers to a process of periodically separating cells and transferring them to fresh medium to prevent cell death caused by excessive confluence or nutrient depletion and to maintain their proliferative capacity over an extended period. During this process, the cells may maintain their fundamental morphological characteristics and surface marker expression patterns while undergoing continuous division, and cells subjected to a certain number of passages may stably adapt to the culture environment and exhibit a uniform proliferation pattern. Accordingly, subcultured cells may exhibit improved experimental reproducibility compared to initially isolated cells and may provide reliable results in various applications, including drug treatment, differentiation induction, and transplantation studies.

In one embodiment of the present invention, the characteristics of the dura mater-derived stem cells of the present invention were confirmed by analyzing marker expression from passage 1 to passage 8, demonstrating that various properties are exhibited and maintained. Accordingly, in one embodiment of the present invention, the dura mater-derived stem cells may be passaged 1 to 10 times, and for example, may be passaged 1 to 9 times or 1 to 8 times, but are not limited thereto.

In one embodiment of the present invention, a cell of the neural lineage may be characterized by one or more selected from the following, but is not limited thereto:
increased axon and dendrite formation in neurons; and
increased protrusion density and formation in neurons.

In one embodiment of the present invention, it was confirmed that, when FBS was used in α-MEM medium generally used as a cell culture medium, dura mater-derived stem cells exhibiting superior differentiation potency and a higher likelihood of maintaining cell viability upon subsequent transplantation could be produced compared to when FBS was used in IMDM medium. At this time, it was confirmed that even when GRO was used instead of FBS, the cells exhibited similar size while maintaining excellent proliferative capacity.

Accordingly, the present invention provides a medium composition for producing dura mater-derived stem cells, comprising as active ingredients:
a basal medium composition including one or more active ingredients selected from the group consisting of amino acids, vitamins, minerals, glucose, and cell culture supplements; and
serum or a serum substitute.

In the present invention, the term “basal medium composition” may refer to a composition that provides a fundamental environment for cell survival and proliferation by including basic nutrients such as essential amino acids, vitamins, inorganic salts, glucose, and cell culture supplements. In particular, in the present invention, as a medium composition serving as the basis for preparing stem cells from the dura mater, the medium composition for producing dura mater-derived stem cells may additionally include serum or a serum substitute in the basal medium composition.

In one embodiment of the present invention, the amino acid may be one or more selected from the group consisting of L-alanine, L-arginine, L-asparagine, L-aspartic acid, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine:
the vitamin may be one or more selected from the group consisting of ascorbic acid, biotin, choline chloride, folic acid, inositol, nicotinamide, calcium pantothenate, pyridoxine-HCl, riboflavin, thiamine-HCl, and vitamin B12;
the inorganic salt may be one or more selected from the group consisting of sodium chloride, potassium chloride, calcium chloride, magnesium sulfate, sodium dihydrogen phosphate, and sodium bicarbonate;
the glucose may be D-glucose; and
the cell culture supplement may be adenine sulfate, but is not limited thereto.

In the present invention, the “medium composition for producing dura mater-derived stem cells,” which additionally includes serum or a serum substitute in the basal medium composition, may fall within the scope without limitation so long as it is characterized by containing the above components. In one embodiment of the present invention, α-minimum essential medium (α-MEM) was used as an example thereof. In the present invention, “α-minimum essential medium (α-MEM)” may refer to a medium formulated by modifying the conventional Eagle's MEM to optimize the ratios of essential metabolic components such as amino acids, vitamins, inorganic salts, and glucose required for cell culture. This medium is particularly advantageous for maintaining cell viability and preserving cell cycle stability and differentiation potency during long-term culture of various progenitor cell populations, including mesenchymal stem cells, fibroblasts, and neural progenitor cells. Additionally, α-MEM includes adenine sulfate, which supports nucleotide synthesis pathways, and contains phenol red as an indicator component, thereby enabling monitoring of pH changes in the culture environment and serving as a medium that may secure both experimental reproducibility and culture efficiency. α-MEM may be understood to be compatible with the “basal medium composition” of the present invention, in that α-MEM essentially includes amino acids, vitamins, inorganic salts, glucose, and cell culture supplements. Furthermore, since α-MEM may additionally contain phenol red as an indicator, it is evident that the basal medium composition of the present invention may further include phenol red.

In the present invention, the term “serum” refers to the supernatant obtained after blood coagulation and may promote cell survival and proliferation by providing growth factors, hormones, proteins, lipids, and inorganic ions during cell culture.

Additionally, in the present invention, the term “serum replacement” may refer to a supplement that replaces animal-derived serum components with a chemically defined composition and stably supplies nutrients and growth factors required for cell culture.

In one embodiment of the present invention, the serum may be one or more selected from the group consisting of fetal bovine serum (FBS), newborn calf serum (NCS), calf serum (CS), horse serum, goat serum, donor bovine serum (DBS), and human serum; and the serum substitute may be one or more selected from the group consisting of a growth factor reduced supplement, a chemically defined lipid concentrate, an insulin-transferrin-selenium supplement, a B27 supplement, an N2 supplement, a serum substitute supplement, and a xeno-free serum replacement, but is not limited thereto.

Each serum substitute may include any substance referred to by the corresponding term and may include variants thereof, and is not limited to any specific trademark or product name.

In one embodiment of the present invention, it was confirmed that even when stem cells were produced from the dura mater using a medium composition treated with FBS as the serum or GRO as the serum substitute, the cells expressed a unique combination of markers and exhibited a size advantageous for transplantation. Accordingly, the dura mater-derived stem cells of the present invention may generally be obtained even when the use of serum is replaced with a serum substitute.

The present invention provides a method of producing dura mater-derived stem cells, comprising the step of producing dura mater-derived stem cells by chopping isolated dura mater tissue and culturing the chopped dura mater tissue in the medium composition for producing dura mater-derived stem cells.

In the present invention, the dura mater-derived stem cells produced by the above method may be at passage 0.

The production method of the present invention may further include a step of culturing the dura mater-derived stem cells in the basal culture composition of the present invention, and a step of additionally culturing the dura mater-derived stem cells in α-MEM containing 1% FBS and gentamicin solution, but is not limited thereto.

The present invention provides a kit for producing dura mater-derived stem cells, comprising the medium composition for producing dura mater-derived stem cells and an instruction manual.

In one embodiment of the present invention, the instruction manual may teach the production method, but is not limited thereto.

In the present invention, the term “kit” refers to a tool that enables the production of dura mater-derived stem cells by including the medium composition for producing dura mater-derived stem cells of the present invention. The kit of the present invention may further include, in addition to the above materials, other components, compositions, solutions, or devices that are conventionally required for the storage or handling thereof. As specific examples, each component may be applied one or more times without limitation on the number of applications, there is no limitation on the order in which each material is applied, and the application of each material may occur simultaneously or may occur sequentially.

In the present invention, the kit may include a container, instructions, and the like. The container may serve to package the above substance, and may also serve to store and secure it. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

In the present invention, when the term “comprising” is used, it means that, unless specifically stated otherwise, other components are not excluded but may be further included. Throughout the present invention, the expression “a step of ˜” or “the step of ˜” does not mean “a step for ˜.”

Hereinafter, preferred examples are presented to help understand the present invention. However, the following examples are provided only to help understand the present invention more easily, and the contents of the present invention are not limited by the following examples.

### EXAMPLES

### Example 1. Production of Human Dura Mater-Derived Stem Cells

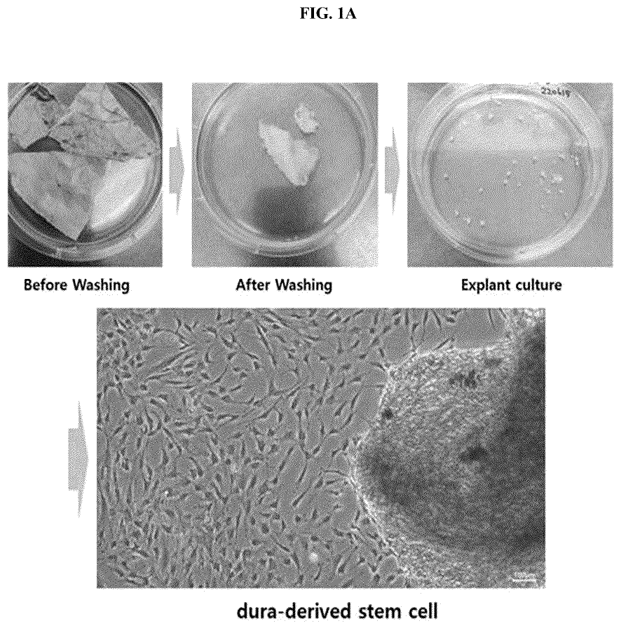

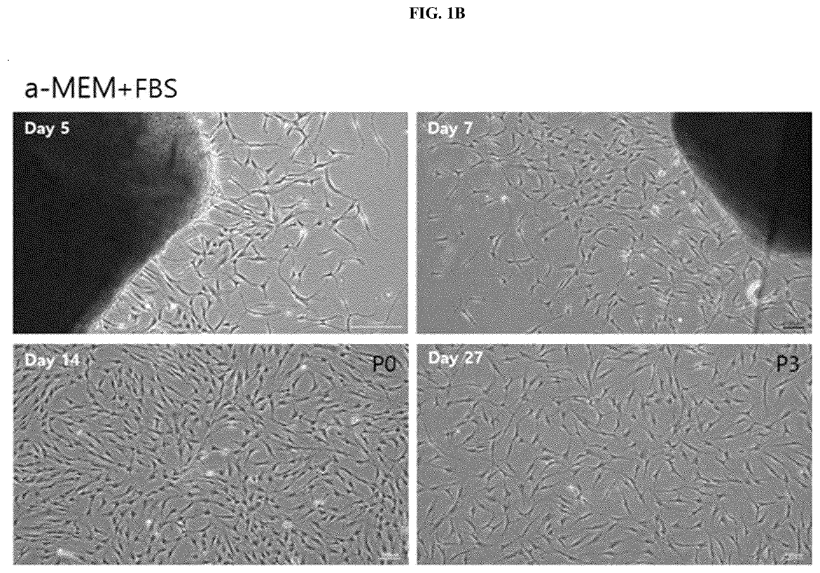

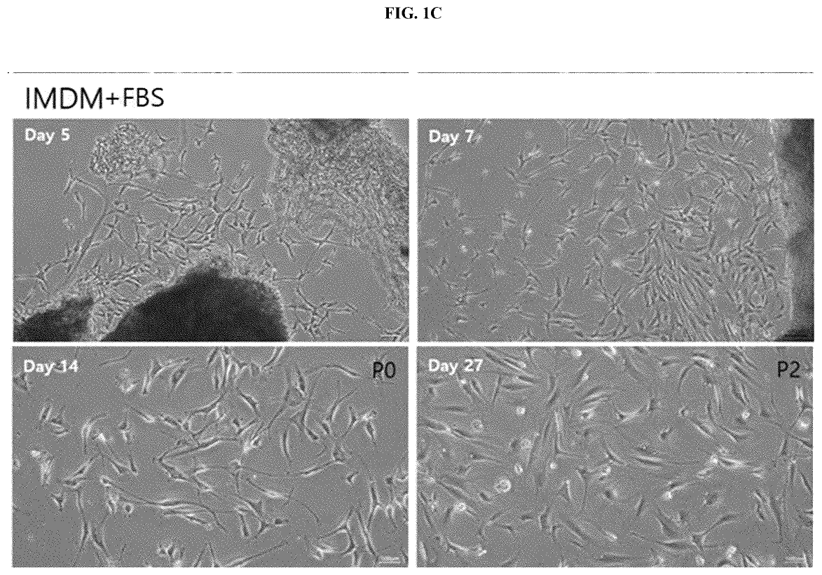

Human dura mater-derived stem cells were produced by isolating cells from the human dura mater using an explant culture method (FIG. 1). First, human dura mater cells were prepared. Specifically, human dura mater tissue was chopped as small as possible into a size of 1 mm³. Forceps were used to divide the tissue into 2 mm diameter lengths, and the tissue was placed on a 100 mm cell culture dish and covered with a cover glass. Minimum Essential Medium (MEM) Alpha (α-MEM, GIBCO, cat. no. 12571-071) containing 10% fetal bovine serum (FBS, GIBCO, cat. no. 16000-044) and 0.1% gentamicin solution (Sigma, cat. no. G1397) was added, and cells were cultured in an incubator under conditions of 37° C. and 5% CO₂. On day 14, passage 0 was confirmed, and dura mater cells were obtained by filtering using a strainer 40 μm (Falcon, cat. no. 352340). The cells were then cultured in a 100 mm cell culture dish using α-MEM. The obtained dura mater cells were again cultured using α-MEM containing 10% FBS and 0.1% gentamicin solution. Subculture was performed using a 100 mm cell culture dish, which was incubated in an incubator under conditions of 37° C. and 5% CO₂. Subculture was performed until the finally cultured cell concentration in each 100 mm cell culture dish was 7×10⁵ cells/mL or more and the cell confluency was 90% to 95%. After stocking at P6, the cells were used in all experiments. In addition, human dura mater-derived stem cells were prepared using the same method using Iscove's Modified Dulbecco's Medium (IMDM) instead of α-MEM.

According to FIGS. 1B and 1C, different cell sizes and cell membrane morphologies were observed depending on the culture medium. Accordingly, in the examples below, the characteristics of the dural membrane-derived stem cells isolated according to each medium were analyzed to establish culture conditions.

### Example 2. Confirmation of the Superior Proliferative Capacity of Human Dura Mater-Derived Stem Cells and Cell Size Analysis

First, the proliferative capacity of human dura mater-derived stem cells was analyzed. This analysis was performed according to the same method as in Example 1, but the experimental groups were set up into four groups as follows. Specifically, when culturing using α-MEM or IMDM medium from passage 1 to passage 6, 10% FBS, which is commonly used, was treated to prepare an α-MEM+FBS group and an IMDM+FBS group. In addition, an α-MEM+GRO group and an IMDM+GRO group were prepared by culturing in each medium using UltraGRO (hereinafter GRO, HELIOS, cat. no. HPCHXCGL 10), which does not contain animal-derived growth factors. At this time, the α-MEM+FBS group was prepared based on a total volume of 500 ml of α-MEM (450 ml)+10% FBS (50 ml) medium at a volume ratio of 9:1. The α-MEM+5% GRO group (or α-MEM+GRO) was prepared based on a total volume of 500 ml of α-MEM (475 ml)+10% GRO (25 ml) medium at a volume ratio of 9.5:0.5.

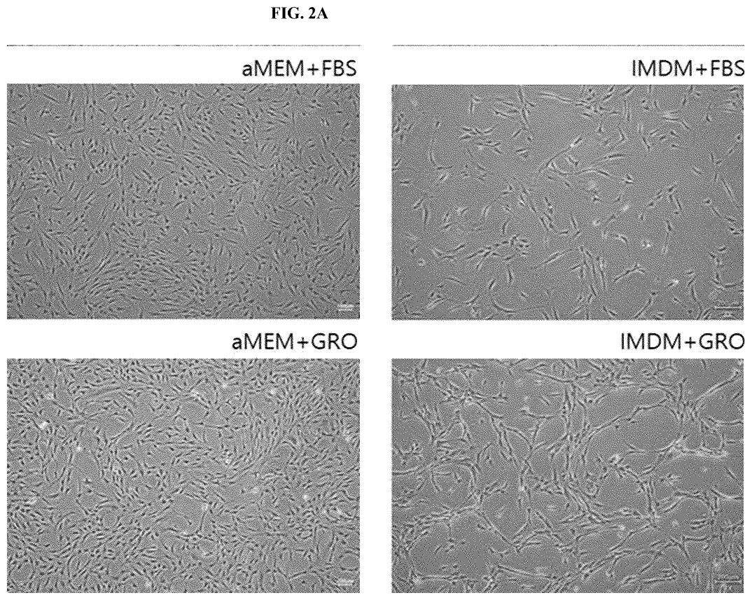

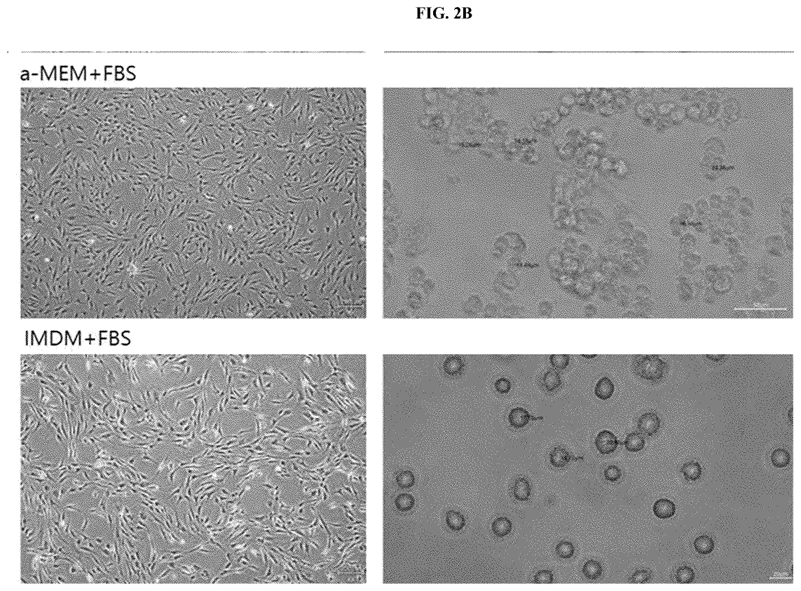

As a result (FIG. 2A), both groups exhibited stable proliferation without morphological or size changes. Accordingly, it was confirmed that the cells according to the present invention may proliferate stably even under xeno-free conditions, supporting their potential use as cell therapy agents.

Meanwhile, when the attached cells were suspended and the single cell size was measured (FIG. 2B), the average size was 16 μm in the α-MEM+FBS group and 18 μm in the IMDM+FBS group, indicating that the cell size in the α-MEM group was smaller. The larger the cell size, the higher the possibility that the cell may be damaged due to the pressure applied by the syringe needle, nozzle, or the like used for transplantation through vascular grafting and cell printing. Therefore, it was inferred that α-MEM is more suitable as a culture medium than IMDM because utilizing smaller cells would maintain cell viability.

### Example 3. Confirmation of Excellent Stability of Human Dura Mater-Derived Stem Cells

### Example 3-1. Low Immune Rejection Response

The immune rejection response of human dura mater-derived stem cells isolated and prepared in α-MEM or IMDM media according to the method described in Example 1 was analyzed. Specifically, human dura-membrane-derived stem cells cultured in each medium were subjected to fluorescence activated cell sorting (FACS) analysis using a human leukocyte antigen (HLA)-DR marker.

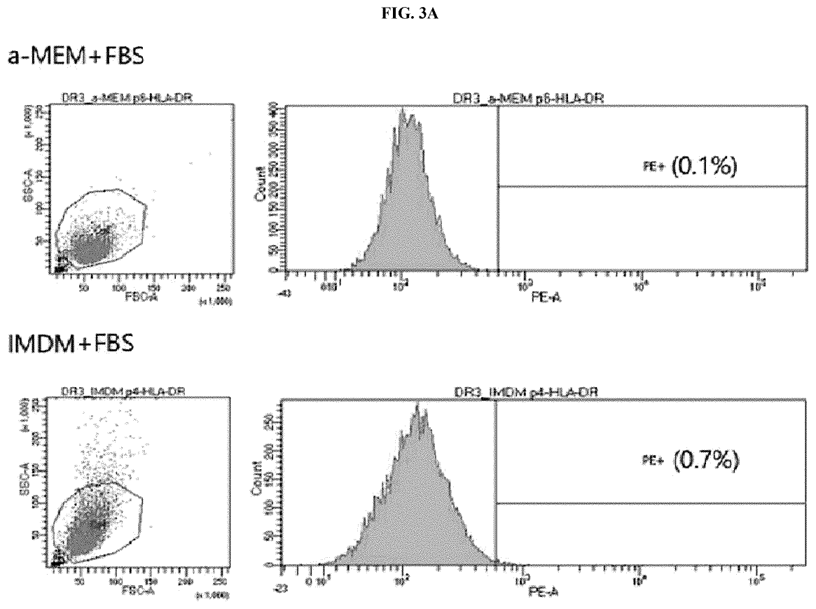

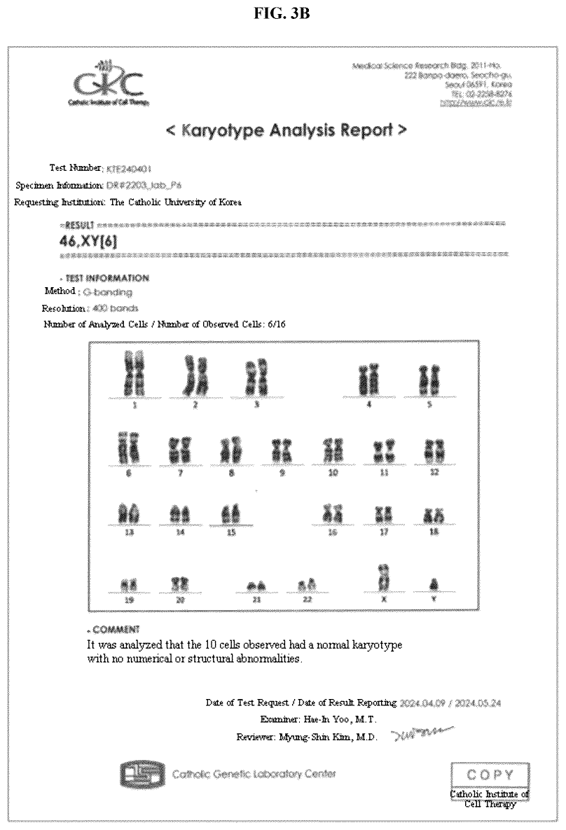

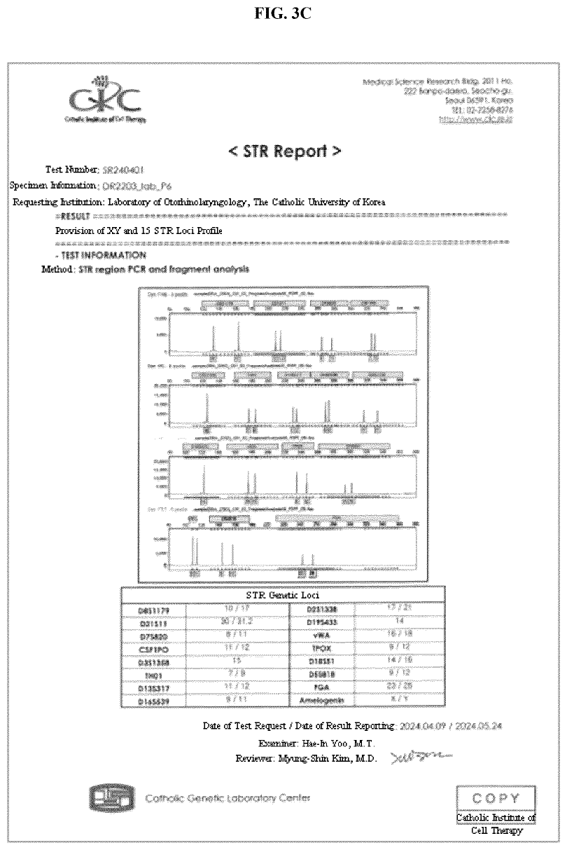

As a result (FIG. 3A), it was confirmed that the HLA-DR marker was hardly expressed, with an expression level of less than 1% in human dura mater-derived stem cells that were produced using each medium. According to these experimental results, it was proven that the human dura mater-derived stem cells produced in Example 1 may be utilized as a cell therapeutic agent.

### Example 3-2. Excellent Genetic Stability

The genetic stability of human dura mater-derived stem cells isolated and prepared in the α-MEM+FBS medium according to the method of Example 1 was analyzed.

First, a karyotype analysis was performed. Specifically, cells were cultured in 100 mm culture dishes until they reached 70% confluency. Next, a colcemid solution was added at a concentration of 10 μg/ml according to the amount of culture medium, and the cells were cultured in an incubator for 3 hours and 30 minutes under conditions of 37° C. and 5% CO₂. After washing with Dulbecco's phosphate buffered saline (DPBS), 1 ml of 0.25% trypsin-ethylenediaminetetraacetic acid (hereinafter 0.25% trypsin-EDTA, GIBCO, cat. no. 25200-056) was added, and the cells were harvested by 0.25% trypsin-EDTA enzyme treatment under conditions of 37° C. and 5% CO₂. The harvested cells were treated with 1 ml of preheated 0.075 mol/L KCl for 30 minutes in a water bath at 37° C. Thereafter, the cells were washed and fixed with Carnoy's fixative solution, and the cell fixative solution was drop-stained on slides. After drying at 90° C. for 40 minutes, the slides were stained with Giemsa solution. Images were acquired by scanning with a Metafer slide scanning system, and the captured images were then analyzed using Ikaros analysis software.

A genetic stability (short tandem repeat (STR)) analysis was performed as described below. Specifically, a pre-mixer was prepared. The pre-mixer was prepared by mixing 10.5 μl of AmpFLSTR PCR Reaction Mix, 0.5 μl of AmpliTaq Gold DNA polymerase, and 5.5 μl of AmpFLSTR Identifiler Primer Set. Thereafter, 15 μl of the pre-mixer, 9 μl of sterile distilled water (DW), and 1 μl of 2 ng DNA were dispensed into a 0.2 μl tube to prepare a reaction mixture having a total volume of 25 μl. However, when using a control (Control DNA), 12 μl of pcDNA was dispensed instead of DW to prepare a mixture having a total volume of 25 μl. Next, pre-denaturation was performed at 95° C. for 11 minutes using the prepared mixture, and then the PCR reaction was performed for 28 cycles of 94° C. for one minute, 59° C. for one minute, and 72° C. for one minute, and finally, 60° C. for 60 minutes. After the PCR reaction was completed, 0.8 μl was taken from each sample and dispensed into a new 0.2 μl tube along with 0.2 μl of size standard and 10 μl of Hi-Di Formamide. In addition, one allelic ladder was prepared in parallel for every 15 samples, and the allelic ladder was prepared by mixing 1 μl of ladder, 0.5 μl of size standard, and 10 μl of Hi-Di Formamide. The prepared samples and the allelic ladders were subjected to a denaturation reaction at 95° C. for three minutes, and then were immediately allowed to stand at 4° C. for five minutes. Finally, the sample that completed the above-described denaturation reaction was injected into ABI 3100 sequencer equipment and analyzed to read the base sequence.

As a result of analyzing genetic stability through karyotype and STR using the above-described method (FIGS. 3B and 3C), it was confirmed that all human dura mater-derived stem cells isolated and produced in Example 1 exhibited suitable stability.

### Example 4. Establishment of Culture Conditions for Human Dura Mater-Derived Stem Cells

In Example 1, the expression markers of human dura mater-derived stem cells isolated and produced under conditions in which FBS was added to α-MEM or IMDM medium were analyzed by FACS. Specifically, the cultured cells were harvested, suspended in an appropriate buffer, and allowed to react with a fluorescently labeled antibody that specifically binds to the marker of interest (Table 1). As needed, an isotype control antibody was included to confirm the background signal. Thereafter, the cells were washed to remove nonspecific binding and injected into a flow cytometer to measure each fluorescent signal. From the measured data, the fluorescence intensity of each cell group was quantified by multichannel analysis software to evaluate the occurrence and level of expression of specific stem cell markers.

TABLE 1

| no. | Marker/antibody | Antibody Information |
|---|---|---|
| 1 | CD9 | BD Pharmingen ™ FITC Mouse Anti-Human |
| | | CD09/cat. no. 555371 (1:50) |
| 2 | CD44 | BD Pharmingen ™ FITC Mouse Anti-Human |
| | | CD44/cat. no. 555479 (1:50) |
| 3 | CD106 | BD Pharmingen ™ PE Mouse Anti-Human |
| | | CD106/cat. no. 551146 (1:50) |
| 4 | CD166 | BD Pharmingen ™ PE Mouse Anti-Human |
| | | CD166/cat. no. 559263 (1:50) |

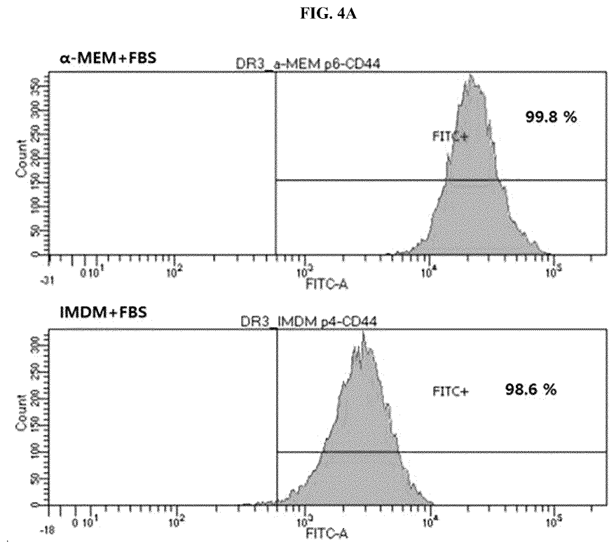

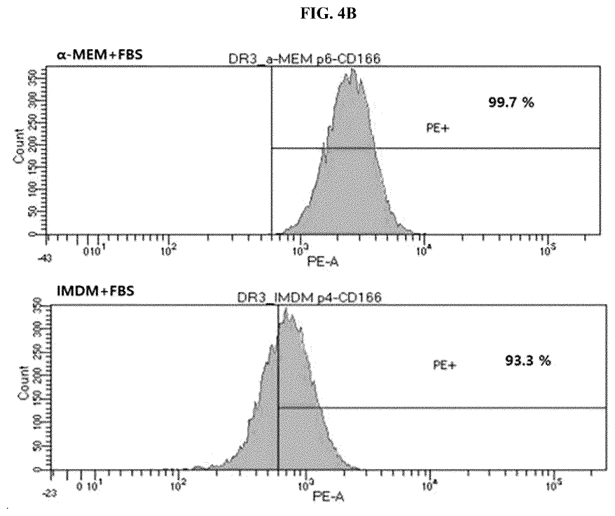

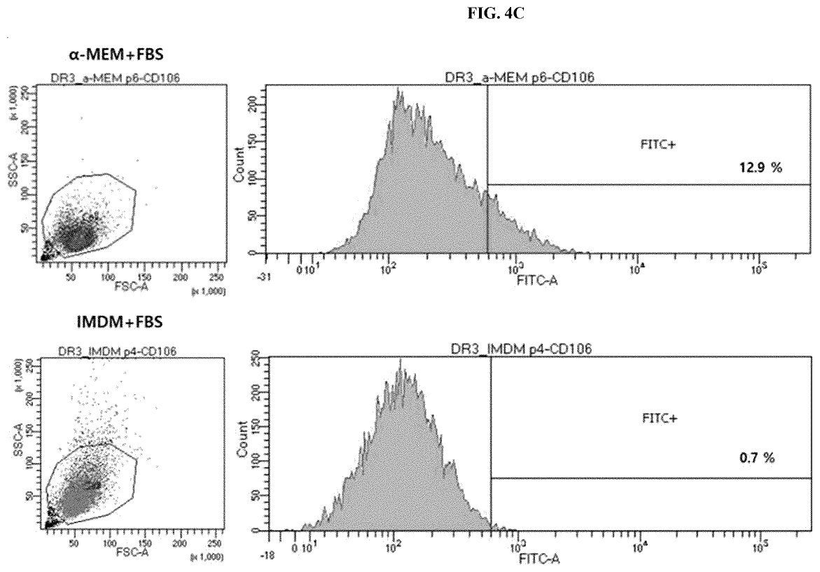

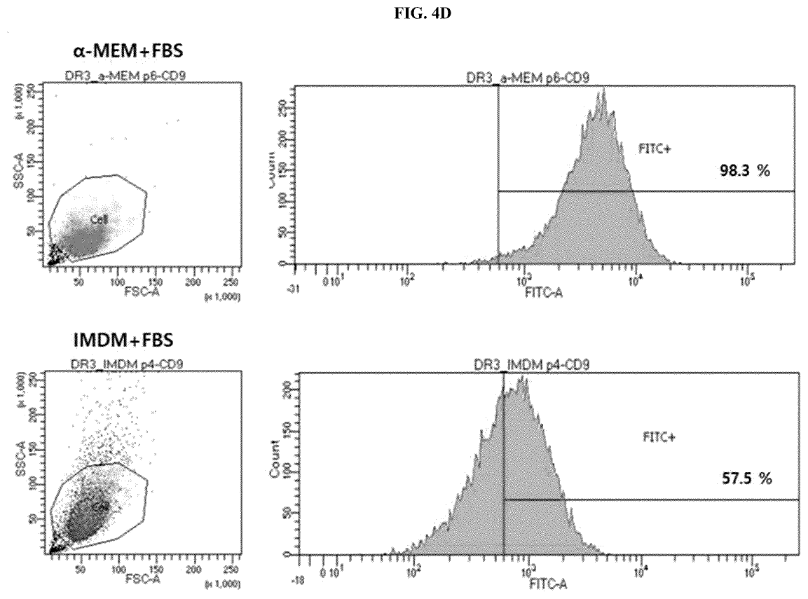

As a result, the results obtained by confirming the expression with the MSC markers of the cells cultured in the two media are shown in FIGS. 4A and 4B. Specifically, both α-MEM+FBS and IMDM+FBS were found to express the MSC markers CD44 and CD166. In particular, α-MEM+FBS was confirmed to exhibit a higher expression level for each marker compared to IMDM+FBS.

Additionally, the expression of endothelial cell and embryonic stem cell (or induced pluripotent stem cell) markers was analyzed. At this time, the expression of CD106, which is used as an endothelial cell marker, and CD9, known as a marker of embryonic stem cells (or induced pluripotent stem cells) were analyzed.

As a result (FIGS. 4C and 4D), it was observed that each marker exhibited a significantly higher level of expression under the medium condition of α-MEM+FBS, confirming that that the differentiation potency toward various markers was superior in the medium.

In summary, in Example 2, the cell size of the α-MEM+FBS group was smaller than that of the IMDM+FBS group, suggesting that the α-MEM+FBS group may be more advantageous for maintaining cell viability during future vascular transplantation and cell printing. Furthermore, in Example 4, the expression levels of specific markers in various fields were found to be higher in the α-MEM+FBS group, confirming that a wide range of differentiation potency can be maintained. Therefore, the α-MEM+FBS group was established as a medium condition that enables the development of cell therapeutic agents suitable for transplantation procedures while maintaining a wide range of differentiation potency.

In the following experiments, human dura mater-derived stem cells isolated under the above-described conditions were used.

### Example 5. Characterization of Human Dura Mater-Derived Stem Cells: In-Depth Analysis of Mesenchymal Stem Cell (MSC) Expression Markers and Confirmation of Reproducibility

In this example, negatively or positively expressed MSC-related markers were analyzed to further characterize the human dura mater-derived stem cells of the present invention. The analytical method was the same as in Example 4-2, and the markers analyzed are listed in Table 2.

TABLE 2

| no. | Marker/antibody | Antibody Information |
|---|---|---|
| 1 | IgG1, κ Isotype | BD Pharmingen ™ PE Mouse IgG1, κ Isotype |
| | | Control/cat. no. 555749 (1:50) |
| 2 | CD14 | BD Pharmingen ™ PE Mouse Anti-Human |
| | | CD14/cat. no. 555398 (1:50) |
| 3 | CD19 | BD Pharmingen ™ PE Mouse Anti-Human |
| | | CD19/cat. no. 555413 (1:50) |
| 4 | CD73 | BD Pharmingen ™ PE Mouse Anti-Human |
| | | CD73/cat. no. 550257 (1:50) |
| 5 | CD90 | BD Pharmingen ™ PE Mouse Anti-Human |
| | | CD90/cat. no. 555596 (1:100) |
| 6 | CD105 | BD Pharmingen ™ PE Mouse Anti-Human |
| | | CD105/cat. no. 560839 (1:50) |
| 7 | HLA-DR | BD Pharmingen ™ PE Mouse Anti-Human |
| | | HLA-DR/cat. no. 556644 (1:50) |
| 8 | CD106 | BD Pharmingen ™ PE Mouse Anti-Human |
| | | CD106/cat. no. 551146 (1:50) |
| 9 | CD29 | BD Pharmingen ™ PE Mouse Anti-Human |
| | | CD29/cat. no. 556049 (1:50) |
| 10 | IgG1, κ Isotype | BD Pharmingen ™ FITC Mouse IgG1, κ |
| | | Isotype Control/cat. no. 555748 (1:50) |
| 11 | CD9 | BD Pharmingen ™ FITC Mouse Anti-Human |
| | | CD09/cat. no. 555371 (1:50) |

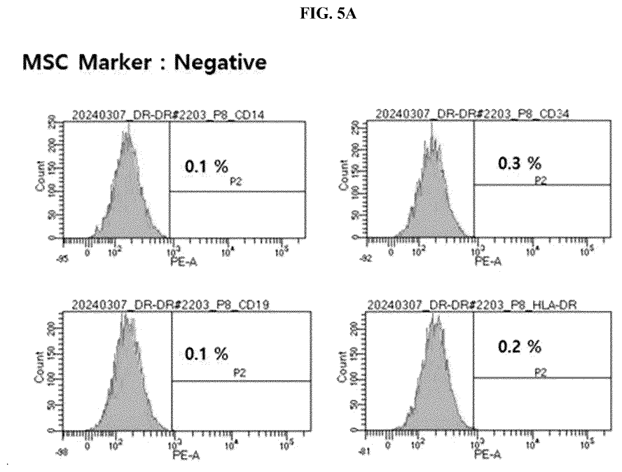

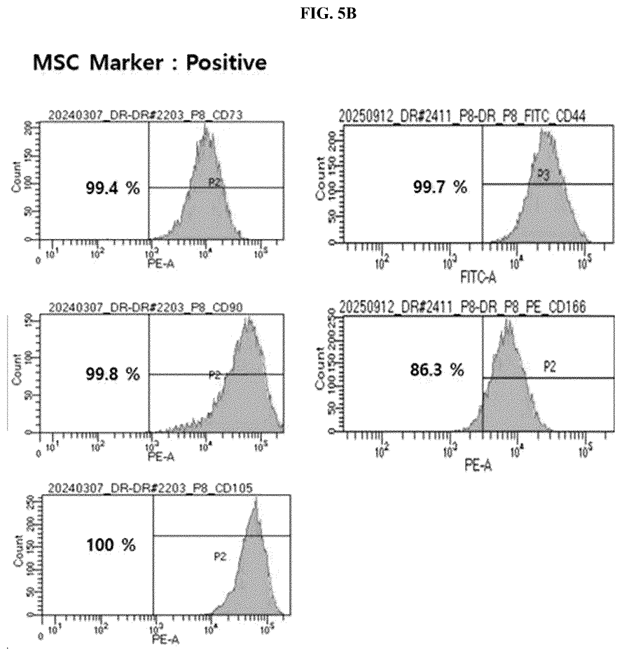

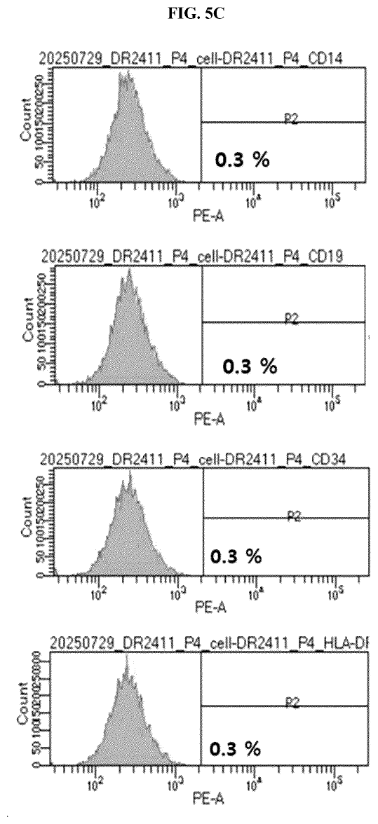

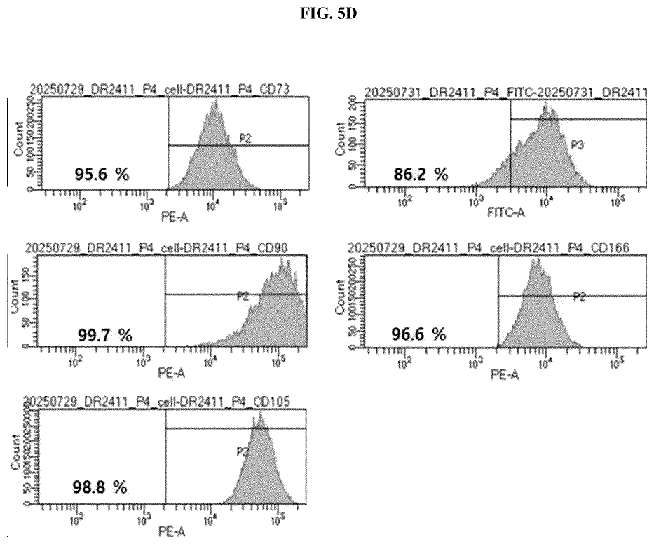

As a result, after culturing cells up to P8, the negative and positive expression patterns for MSC markers were confirmed as shown in FIGS. 5A and 5B, respectively. Specifically, negative makers were CD14, CD19, CD34, and HLA-DR, all of which were measured to be less than 1%. In addition, positive makers were CD44, CD73, CD90, CD105, and CD166, all of which were measured to be more than 99%. According to these experimental results, the human dura mater-derived stem cells of the present invention exhibited all the characteristics of native MSCs, and these characteristics were maintained up to P8, confirming that they have excellent reproducibility and are suitable as a cell therapy agent.

In addition, when reproducibility was confirmed with FACS markers in P4 and P6 of the dura mater-derived stem cells of another donor isolated and cultured in the same manner, it was proven that the cells had the same unique marker expression characteristics (FIGS. 5C to 5E).

### Example 6. Confirmation of Excellent Proliferative Capacity and Post-Thawing Cell Viability of Human Dura Mater-Derived Stem Cells

In this example, the proliferative capacity and post-thawing cell viability of human dura mater-derived stem cells were analyzed. To this end, a CCK-8 assay was performed to determine cell proliferation in P6 cells, which have potential for cell therapeutic agent development. Specifically, for cell inoculation, cells were plated at 1.5×10³ cells/well in a 48-well plate and adherently cultured for 24 hours. For the CCK-8 assay, Cell Counting Kit-8 (Dojindo Laboratories, Japan, cat. no. CK04) and a multi-function ELISA Reader (Spectramax M2e multi plate reader, USA) were prepared. 10 μL of CCK-8 reagent per 100 μL of medium was added to each well and gently shaken to mix evenly, and cells were incubated in an incubator for two hours under conditions of 37° C. and 5% CO₂. Thereafter, the absorbance (optical density (OD)) was measured at 450 nm using the microplate reader (multi-function ELISA Reader (Spectramax M2e multi plate reader, USA)).

Meanwhile, for post-thawing stability testing, cell viability was measured at 1 month, 2 months, 6 months, 10 months, 12 months, and 18 months.

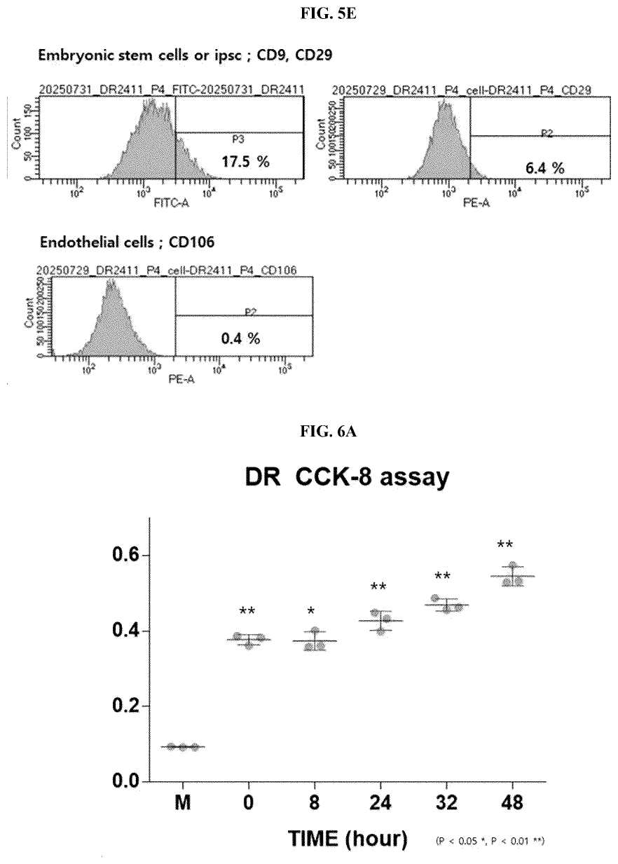

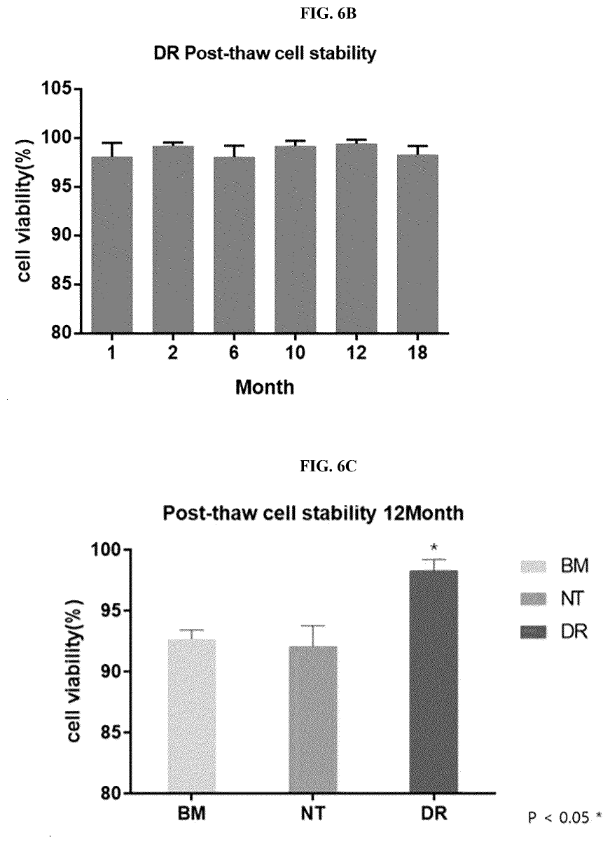

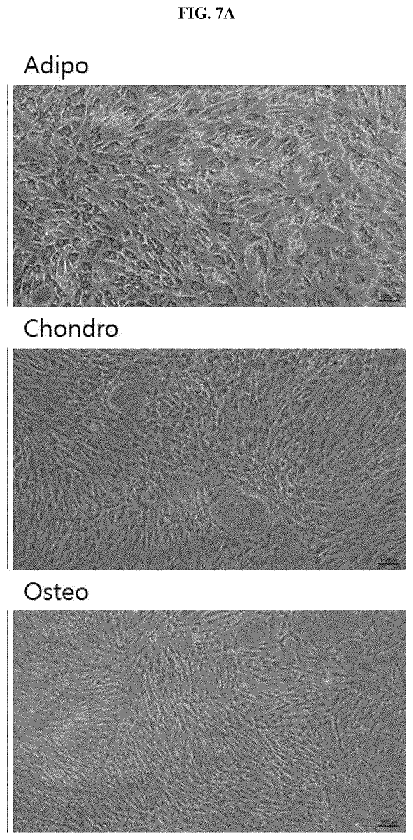

As a result, according to FIG. 6A, P6 cells, which have potential for cell therapeutic agent development, demonstrated superior cell proliferation at a statistically significant level.

In addition, according to FIG. 6B, frozen cells maintained viability for up to 18 months. Furthermore, the results of the same experiment performed on bone marrow (BM) cells and neural (neuron/neural tissue (NT)) cells at 12 months were confirmed, as shown in FIG. 6C. According to these results, the dura mater-derived stem cells of the present invention exhibited superior cell viability compared to BM cells and neurons.

### Example 7. Confirmation of Excellent Differentiation Potency of Human Dura Mater-Derived Stem Cells into Mesodermal and Neural Lineages

In this example, the differentiation potency of human dura mater-derived stem cells into mesodermal and neural lineages was analyzed.

### Example 7-1. Differentiation Potency into Mesodermal Lineage

The method for analyzing differentiation potency into mesodermal lineages is as follows. Specifically, 1.5×10⁴ cells/well were plated into a 2-well chamber plate and adherently cultured for 24 hours. After 24 hours, the differentiation medium was replaced, and the medium was then replaced with fresh medium every two to three days for three weeks. The differentiation medium used was StemPro™ Adipogenesis Differentiation Kit [Cat. no. A1007001], StemPro™ Chondrogenesis Differentiation Kit [Cat. no. A1007101], and StemPro™ Osteogenesis Differentiation Kit [Cat. no. A1007201]. To confirm differentiation into mesoderm, differentiation into three representative types of mesodermal lineage cells, which are adipocytes, chondrocytes, and osteoblasts/osteocytes, was confirmed.

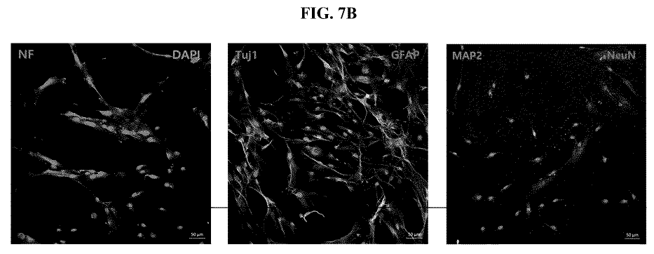

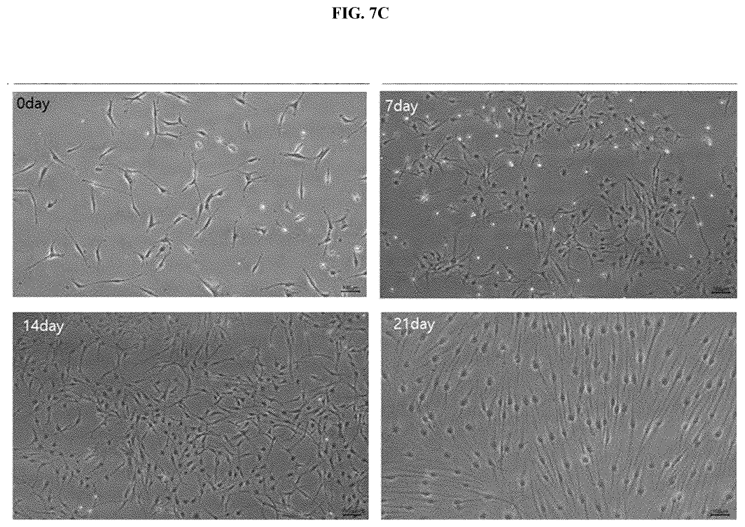

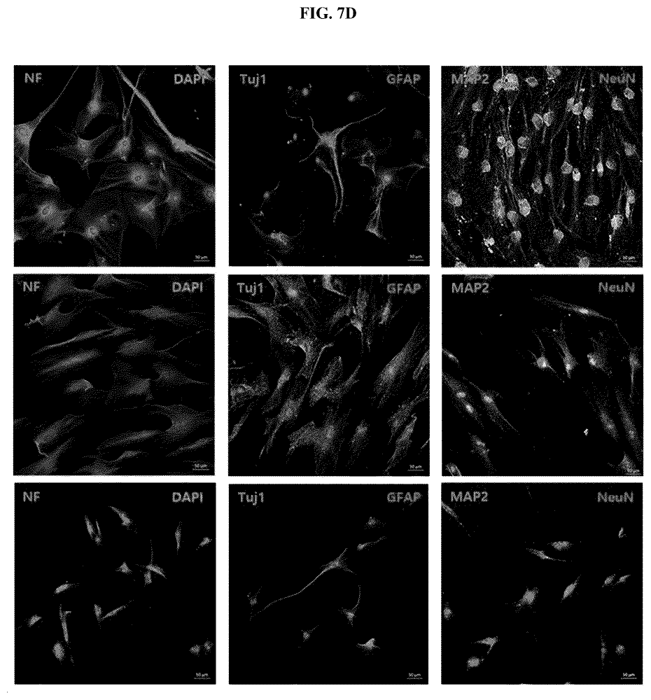

As a result, according to FIG. 7A, the morphological features of adipocytes, chondrocytes, and osteocytes were observed as the human dura mater-derived stem cells were differentiated and cultured, and thus it was confirmed that the human dura mater-derived stem cells of the present invention have excellent differentiation potency into mesodermal lineages.

### Example 7-2. Differentiation into Neural Lineage

Neurofilament (NF), neuron-specific class III β-tubulin (Tuj1), glial fibrillary acidic protein (GFAP), microtubule-associated protein 2 (MAP2), and neuronal nuclei (NeuN) are all differentiation stage and cell subtype-specific markers of neurons, so they are important indicators for determining how cells differentiate into the nervous system. Therefore, to analyze the differentiation potency into neural lineage cells, the expression levels of NF (Neurofilament-Mouse, Abcam, cat.no. ab24575), Tuj1 (Neuron-specific class III β-tubulin-Mouse, Abcam, cat.no. ab11315), GFAP (Glial Fibrillary Acidic Protein-Rabbit, Abcam, cat.no. AM5804), MAP2 (Microtubule-Associated Proteins-Mouse, Sigma, cat.no. M9942), and NeuN (Neuronal Nuclei-Rabbit, Abcam, cat.no. ab104225) were analyzed.

As a result, in order to confirm the differentiation stage of neurons of the dura mater-derived stem cells before differentiation, the results of staining with markers of NF, TuJ1, GFAP, MAP2, and NeuN before differentiation were confirmed as shown in FIG. 7B. Specifically, cells expressing a small amount of NF, TuJ1, and GFAP were included. In addition, it was confirmed that MAP2 and NeuN were not expressed. These results mean that most of the isolated cell population was undifferentiated or in a progenitor cell state. In addition, only a very small number of neurons expressed NF, TuJ1, and GFAP, confirming that they were in a glial cell state, and since mature neuron markers such as MAP2 and NeuN were not expressed, it was confirmed that they were in a stage before differentiation into functional neurons.

In addition, FIG. 7C shows that the morphology of cells changed during neural differentiation at day 0, day 7, day 14, and day 21. According to this, differentiation into neurons was observed for 21 days, confirming that neural differentiation potency was expressed within a short period of time.

### Example 7-3. Superior Neural Differentiation Potency Compared to Stem Cells Derived from Other Tissues

Inferior turbinate-derived stem cells and bone marrow-derived stem cells were used as control groups, and the expression of neuron-specific markers before and after neural differentiation was further analyzed. The experimental method was the same as in Example 7-2.

The results are shown in FIG. 7D. From top to bottom, the expression of corresponding markers for dura mater-derived stem cells, inferior turbinate-derived stem cells, and bone marrow-derived stem cells is shown in respective rows.

First, as a result of observing the size of each type of stem cells after differentiation into neurons, a significant number of dura mater-derived stem cells exhibited larger growth than inferior turbinate-derived stem cells and bone marrow-derived stem cells. In addition, dura mater-derived stem cells exhibited strong expression of NF and Tuj1, and many cells exhibited strong expression of MAP2 and NeuN.

These experimental results suggest that dura mater-derived stem cells undergo structural and functional maturation more smoothly during the differentiation process into neurons compared to stem cells from other tissues.

In addition, at the same time, it was observed that neural-specific protrusions (neurites) such as axons and dendrites were distinctly formed and actively extended in the cells, indicating that stable morphological maturation also occurred with neural development. These results suggest that dura mater-derived stem cells not only express neural markers, but also exhibit a greater number of cells having the structural features of actual neurons compared to stem cells derived from other sources when differentiation is induced, and they exhibit active neurite growth and extension. Therefore, dura mater-derived stem cells are considered to have high neuron differentiation efficiency and excellent maturity, and furthermore, they have the potential to form functional neural networks.

The foregoing description of the present invention is intended for illustrative purposes, and it will be understood by those skilled in the art that various modifications can be made thereto in other specific forms without departing from the technical spirit or essential features of the present invention. Therefore, the embodiments described above should be understood as illustrative in all respects and not restrictive.

1. A dura mater-derived stem cell negatively expressing one or more markers selected from the group consisting of CD14, CD19, CD34, and HLA-DR and positively expressing one or more markers selected from the group consisting of CD44, CD73, CD90, CD105, and CD166.

2. The dura mater-derived stem cell of claim 1, wherein the dura mater-derived stem cell further positively expresses one or more selected from the group consisting of CD106 and CD9.

3. The dura mater-derived stem cell of claim 1, wherein the dura mater-derived stem cell has an average diameter of 14 μm to 18 μm.

4. The dura mater-derived stem cell of claim 1, wherein the dura mater-derived stem cell differentiates into one or more mesodermal lineages selected from the group consisting of adipocytes, chondrocytes, and osteocytes, or differentiates into a neural lineage.

5. The dura mater-derived stem cell of claim 4, wherein a cell of the neural lineage positively expresses one or more selected from the group consisting of neurofilament (NF), neuron-specific class III β-tubulin (Tuj1), glial fibrillary acidic protein (GFAP), microtubule-associated protein 2 (MAP2), and neuronal nuclei (NeuN).

6. The dura mater-derived stem cell of claim 1, wherein the dura mater-derived stem cell is passaged 1 to 10 times.

7. The dura mater-derived stem cell of claim 4, wherein a cell of the neural lineage is characterized by one or more of the following:
increased axon and dendrite formation in neurons; and
increased protrusion density and formation in neurons.

8. A medium composition for producing the dura mater-derived stem cells of claim 1, comprising as active ingredients:
a basal medium composition including one or more active ingredients selected from the group consisting of amino acids, vitamins, minerals, glucose, and cell culture supplements; and
serum or a serum substitute.

9. The composition of claim 8, wherein the amino acid is one or more selected from the group consisting of L-alanine, L-arginine, L-asparagine, L-aspartic acid, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine,
the vitamin is one or more selected from the group consisting of ascorbic acid, biotin, choline chloride, folic acid, inositol, nicotinamide, calcium pantothenate, pyridoxine-HCl, riboflavin, thiamine-HCl, and vitamin B12,
the inorganic salt is one or more selected from the group consisting of sodium chloride, potassium chloride, calcium chloride, magnesium sulfate, sodium dihydrogen phosphate, and sodium bicarbonate,
the glucose is D-glucose, and
the cell culture supplement is adenine sulfate.

10. The composition of claim 8, wherein the serum is one or more selected from the group consisting of fetal bovine serum (FBS), newborn calf serum (NCS), calf serum (CS), horse serum, goat serum, donor bovine serum (DBS), and human serum, and
the serum substitute is one or more selected from the group consisting of a growth factor reduced supplement, a chemically defined lipid concentrate, an insulin-transferrin-selenium supplement, a B27 supplement, an N2 supplement, a serum substitute supplement, and a xeno-free serum replacement.

11. A method of producing the dura mater-derived stem cells of claim 1, comprising a step of producing dura mater-derived stem cells by chopping isolated dura mater tissue and culturing the chopped isolated dura mater tissue in the medium composition for producing dura mater-derived stem cells of claim 8.

12. A kit for producing dura mater-derived stem cells, comprising the medium composition for producing dura mater-derived stem cells of claim 8 and an instruction manual.

13. The kit of claim 12, wherein the instruction manual teaches the production method of claim 11.
